(19) United States
(12) Patent Application Publication
(10) Pub. No.: US 2026/0168027 A1
(43) Pub. Date: Jun. 18, 2026

(54) METHODS FOR GENOTYPING GENE-EDITED HEMATOPOIETIC STEM CELLS

(71) Applicant: KAMAU THERAPEUTICS, INC., South San Francisco, CA (US)

(72) Inventors: Sabastian TREUSCH, South San Francisco, CA (US); William MATERN, South San Francisco, CA (US)

(21) Appl. No.: 19/126,572

(22) PCT Filed: Nov. 2, 2023
(86) PCT No.: PCT/US2023/036720

§ 371 Date: May 1, 2025

Related U.S. Application Data
(60) Provisional application No. 63/382,060, filed on Nov. 2, 2022.
(60) Provisional application No. 63/383,768, filed on Nov. 15, 2022.

(51) Int. Cl. C12Q 1/6883

(52) U.S. Cl. CPC C12Q 1/6883; C12Q 2600/156; C12Q 2600/158

(57) ABSTRACT

Provided herein are methods and compositions relating to the use of single cell RNA sequencing to assess gene editing outcomes in transplanted populations of gene edited hematopoietic stem cells.

### CROSS REFERENCE TO RELATED APPLICATIONS

This application claims the benefit of, and priority to, U.S. Provisional Application No. 63/382,060, filed on Nov. 2, 2022, and U.S. Provisional Application No. 63/383,768, filed Nov. 15, 2022, each of which is hereby incorporated herein by reference in its entirety for all purposes.

### FIELD

Provided herein are methods and compositions relating to the assessment of gene editing outcomes in genetically modified stem cells.

### BACKGROUND

Therapeutic gene editing modifies the genomes of individual cells at one or more targeted loci associated with a disease. Given that each human cell has two copies of the genome, gene editing procedures such as those utilizing CRISPR/Cas systems can give rise to various genomic outcomes in a targeted cell population, including cells having no change to the either allele of the target locus, changes to only one allele, or changes to both alleles. In those alleles bearing changes, modifications can include insertions or deletions (INDELS) of one or more nucleotides, or homology-directed repair (HDR)-mediated insertion of donor polynucleotides that aim to address one or more mutation(s) of the targeted locus. Cell populations having undergone the editing procedure can then administered to a patient, for example, a patient whose hematopoietic stem cells were harvested and edited ex vivo.

Sickle cell disease (SCD) is a genetic condition typically caused by a single point mutation at codon 6 in both copies of the beta-globin (HBB) gene, resulting in an E6V mutation that gives rise to sickle(S) hemoglobin (HbS) production instead of adult (A) hemoglobin (HbA). Gene-edited autologous hematopoietic stem cell-based therapies in clinical development for SCD include those that are designed to directly correct the underlying point mutation, thereby decreasing HbS production and restoring HbA expression. Previous allogeneic hematopoietic stem cell transplant studies, mostly in the context of using HLA-matched related donors, has demonstrated a competitive advantage for homozygous (AA) or heterozygous (AS) hemoglobin due to ineffective erythropoiesis of SS erythroid progenitors. However, at early timepoints following infusion of gene corrected HSCs, red blood cells (RBCs) expressing corrected HbA cannot be distinguished from HbA in transfused blood. Because RBCs are enucleated, tracking of gene editing rates at the genomic level is not possible. Therefore, there is a need for methods that can quantify gene editing outcomes at early timepoints following HSC transplantation and engraftment in a patient.

### SUMMARY

The present disclosure provides methods which utilize reticulocytes derived from a recipient of gene-edited hematopoietic stem cells (HSCs) to assess gene editing outcomes in a transplanted gene-edited HSC population. While RBCs derived from a gene-edited HSC are enucleated and thus cannot be utilized for genotyping, reticulocytes are immature RBCs that still contain some RNA which could be used to evaluate allelic correction. The methods described herein utilize single-cell RNA sequencing (scRNAseq) to enable enumeration of potential gene-editing outcomes in peripheral reticulocytes. The modified genotypes can then be directly combined with the other single cell measurements to assess if gene editing outcomes are linked to other aspects of cell state, such as cell type, as assigned via RNA or protein surface markers, or chromatin accessibility.

### BRIEF DESCRIPTION OF THE FIGURES

FIG. 1 depicts an exemplary method for precisely correcting the disease-causing mutation in the beta-globin gene to reduce HbS and restore HbA expression. The scissors represents a CRISPR/Cas nuclease generating a double-stranded break at the HBB target locus of a hematopoietic stem cell from a subject having sickle cell disease. A corrective polynucleotide, comprising the sequence GAG encoding glutamic acid and designed to replace the GTG mutation encoding valine at codon 6, is introduced into the cell with the nuclease such that the polynucleotide is integrated into the HBB locus via homology directed repair.

FIG. 2 depicts maturation of a red blood cell (RBC) from a hematopoietic stem cell.

FIG. 3 depicts enrichment and sorting of mixed AA, AS and SS reticulocytes. Isolated reticulocytes from AA blood were distributed across all stages of maturation. The majority of AS reticulocytes were in the late stage of development. Reticulocytes from SS donor samples had the highest purity and were mostly early stage. SS blood contained approximately 10 times more reticulocytes than AA blood. CD, Cluster of Differentiation; FSC-A, Forward Scatter-Area; GPA, Glycophorin-A, RBC, Red Blood Cell; SSC-A, Side Scatter-Area; SSC-H, Side Scatter-Height; TO, Thiazole Orange; WBC, White Blood Cell.

FIG. 4 depicts a single cell RNAseq workflow which captures the 5′ end of HBB transcripts to ensure high-resolution coverage of the E6V mutation, which resides close to the transcription start site in exon 1. HBB, beta-globin; TSO, template switch oligo; UMI, unique molecular identifier.

FIG. 5 depicts an exemplary single cell RNA sequencing and genotyping workflow of the disclosure.

FIG. 6 depicts bulk cDNA sequencing of reticulocytes and blood mixes.

FIG. 7 depicts results of variant calling (either wild type (AA) or sickle disease (SS) in a mixed reticulocyte population using the methods described herein.

FIG. 8 depicts variant allele calls among 1:1:1 mixture of AA, AS and SS reticulocytes.

FIG. 9 depicts mRNA expression profile and t-SNE plots of AA, AS, and SS reticulocytes.

FIG. 10 depicts results demonstrating that SS reticulocytes has higher HBB and HGD2 expression than AA and AS reticulocytes.

FIG. 11 depicts results demonstrating that scRNAseq data from AA, AS and SS reticulocytes can be used to examine genotype-associated gene expression differences.

### DETAILED DESCRIPTION

### Single Cell RNA Sequencing of Reticulocytes From Subjects Receiving Gene Edited HSCs

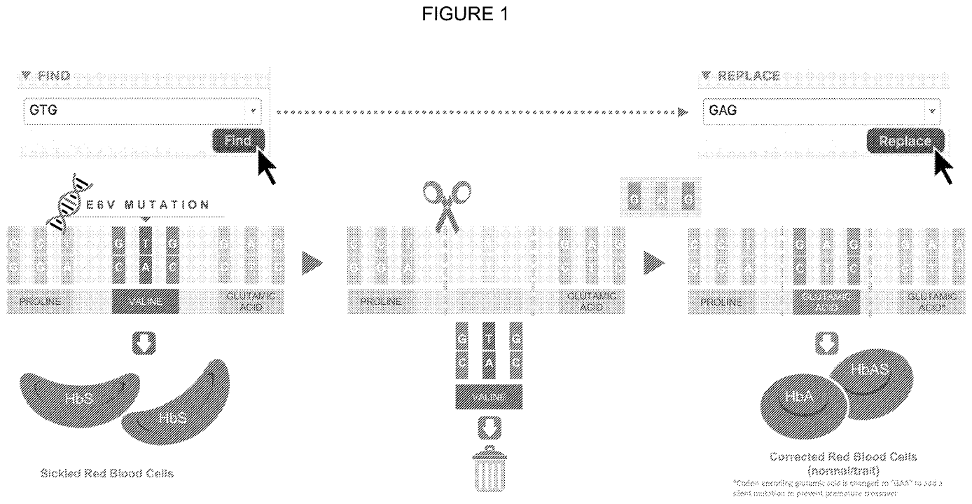

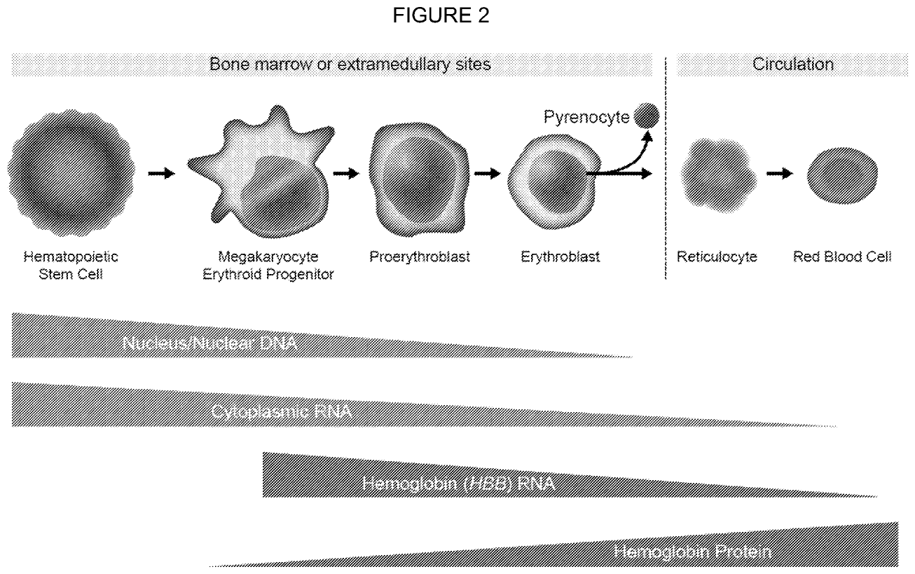

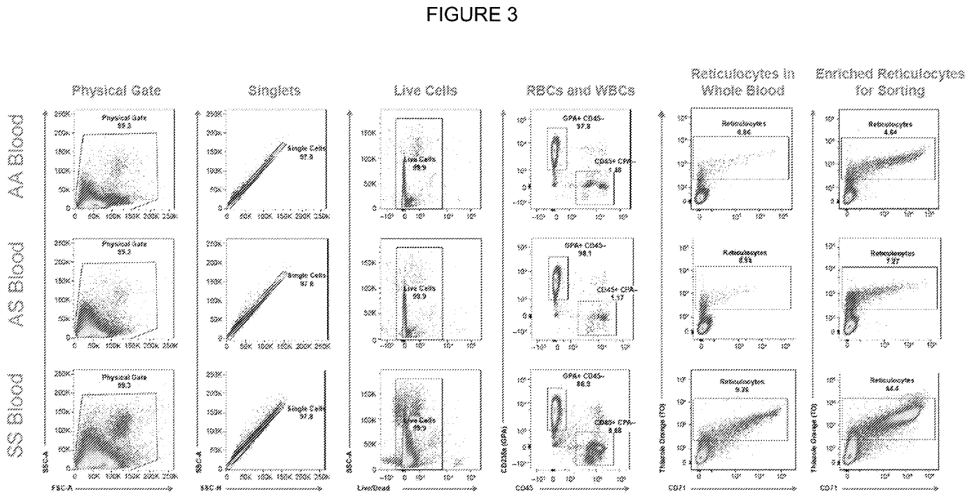

In one aspect, provided herein are methods that can be used to determine the editing status of the hemoglobin (HBB) gene in reticulocytes derived from a patient engrafted with gene-edited HSCs designed to correct the E6V mutation (FIG. 1). The HBB gene is only expressed in the erythroid lineage, which culminates in mature red blood cells that do not contain genomic DNA and are mostly devoid of gene transcripts. Reticulocytes are immature red blood cells that still contain some gene transcripts (FIG. 2). Applying the single cell RNA sequencing and genotyping approach of the disclosure to these cells in particular allows for the assessment of the editing status of the HBB gene, and how this status may influence red blood cell maturation, as well as functional impacts on the expression of other hemoglobin genes. The methods provided herein utilize 10X single cell RNA sequencing. See, e.g., Technical Note, CG000425, ChroumiumNextGEM_SingleCell5′_HT_v2_Reagent, Workflow & Data Overview Rev A, 10x Genomics, (2021, August 9). The 10X method is primarily intended for counting mRNA transcripts to determine gene expression levels, utilizing short sequencing reads to do so. The methods provided herein utilize a modified method capable of longer sequencing reads that can provide sequencing coverage of a gene edit of interest (FIG. 3). The resulting gene of interest-specific sequencing reads are then parsed based on barcodes that tie them to individual cells and analyzed in parallel using Crispresso 2, a software tool intended to analyze editing outcomes based on amplicon sequencing data. See, e.g., https://github.com/pinellolab/CRISPResso2. The Crispresso 2 results are further processed to call the editing outcomes and their zygosity for each barcoded single cell. Single cell editing calls and barcodes are paired and uploaded into the 10X analysis software Loupe. The 10X software can then be utilized to examine connections between single cell editing outcomes and phenotypic differences, such as transcriptional changes that might result from a specific genotype modification.

The methods provided herein can be useful to assess the editing status of any targeted gene of interest in a cell, for example, a genetically modified HSC.

### Gene Editing of HSCs, Transplantation and Engraftment

In certain embodiments, the methods comprise administering to the patient an amount of genetically modified hematopoietic stem cells effective for therapy. In some embodiments of the methods provided herein, the patient is administered an amount of genetically modified hematopoietic stem and progenitor cells effective for therapy. In some embodiments, the administered genetically modified cells can include donor bone marrow cells, umbilical cord blood cells, hematopoietic stem and progenitor cells (HSPCs), peripheral blood CD34⁺ cells, peripheral blood CD34⁺ and CD90⁺ cells, and any combination thereof.

The genetically modified hematopoietic stem cells can be derived from any hematopoietic stem cells deemed useful by the practitioner of skill. In certain embodiments, the genetically modified hematopoietic stem cells, once engrafted, are capable of reconstituting hematopoiesis in the patient. Human hematopoiesis is defined by a cell surface marker expression-based hierarchy initiated by hematopoietic stem cells that both self-renew and differentiate into multipotent progenitors, which in turn give rise to lineage-restricted progenitors, and finally terminally differentiated blood cells (Baum et a., PNAS 89, 2804-2808 (1992); Majeti et al., Cell Stem Cell 1, 635-645 (2007); Doulatov et al., Cell Stem Cell 10, 120-136 (2012)). CD34⁻ expression defines the heterogeneous HSPC population, which can be further classified as a multipotent progenitor (CD34⁺/CD38⁻/CD45RA⁻), long-term repopulating cell in xenograft mice (CD34⁺/CD38⁻/CD90⁺), and a population highly enriched for hematopoietic stem cells (CD34⁺/CD38⁻/CD90⁺/CD45RA⁻).

In certain embodiments, the genetically modified hematopoietic stem cells are of any subtype or colony forming unit. In certain embodiments, the genetically modified hematopoietic stem cells are colony forming unit-granulocyte-erythrocyte-monocyte-megakaryocyte cells. In certain embodiments, the genetically modified hematopoietic stem cells are colony forming unit-erythrocyte cells. In certain embodiments, the genetically modified hematopoietic stem cells are colony forming unit-granulocyte-macrophage cells. In certain embodiments, the genetically modified hematopoietic stem cells are colony forming unit-megakaryocyte cells. In certain embodiments, the genetically modified hematopoietic stem cells are colony forming unit-basophil cells. In certain embodiments, the genetically modified hematopoietic stem cells are colony forming unit-eosinophil cells.

The genetically modified hematopoietic stem cells can be derived from any source deemed useful to the person of skill. In certain embodiments, the hematopoietic stem cells are from a donor. In certain embodiments, the donor is the patient. In certain embodiments, the donor is another subject of the same species, for instance another human. In certain embodiments, the genetically modified hematopoietic stem cells are autologous. In certain embodiments, the genetically modified hematopoietic stem cells are allogeneic. In certain embodiments, the genetically modified hematopoietic stem cells are syngeneic.

The donor hematopoietic stem cells to be genetically modified can be harvested by any technique deemed useful to the person of skill. In some embodiments, the donor subject is administered an hematopoietic stem cells mobilizing agent (e. g plerixafor (Mozobil®), G-CSF, GM-CSF), prior to harvest. In certain embodiments, the hematopoietic stem cells are harvested from peripheral blood. In certain embodiments, the hematopoietic stem cells are harvested from cord blood. In certain embodiments, the hematopoietic stem cells are harvested from bone marrow. In some embodiments, a population of donor cells can be obtained from a product that is collected from a subject, such as a patient or subject in need of an autologous HSCT. The product can be an apheresis product that contains a heterogeneous mixture of cells that have been collected from the subject. The heterogenous mixture of cells can contain primary cells as well as primary CD34+ cells and/or human stem cells and/or progenitor cells (HSPCs). The CD34+ cells and/or HSPCs can be isolated or separated from the other cells in order to obtain a population of stem cells. Following the separation of CD34+ HSPCs, the resulting population of stem cells are substantially free of non-CD34+ cells and are ready for subsequent genetic manipulation.

In some embodiments, the harvested hematopoietic stem cells are separated from the population of primary cells using flow cytometry. In some instances, the flow cytometry comprises fluorescence-activated cell sorting (FACS). In certain other embodiments, the harvested hematopoietic stem cells are separated from the population of primary cells using magnetic bead separation. In some instances, the magnetic bead separation comprises magnetic-activated cell sorting (MACS). In certain other embodiments, the harvested hematopoietic stem cells are separated using a device configured for hematopoietic stem cell enrichment, such as the Miltenyi Biotec CliniMACS cell manufacturing platform.

Methods for culturing or expanding primary hematopoietic stem cells are known in the art, including those described in International Patent Application No. PCT/US2022/72014, which is herein incorporated by reference in its entirety. Methods for culturing primary cells and their progeny are known, and suitable culture media, supplements, growth factors, and the like are both known and commercially available. Typically, human primary cells are maintained and expanded in serum-free conditions. Alternative media, supplements and growth factors and/or alternative concentrations can readily be determined by the skilled person and are extensively described in the literature. In some embodiments, the isolated or purified gene modified cells can be expanded in vitro according to standard methods known to those of ordinary skill in the art.

### Genetically Modified Hematopoietic Stem Cells

In some embodiments, the hematopoietic stem cells are genetically modified to comprise therapeutic heterologous donor polynucleotide sequences. Donor polynucleotide sequences described herein may be incorporated within a wide variety of gene therapy constructs, e.g., to deliver a nucleic acid encoding a protein to a subject in need thereof. A vector construct refers to a polynucleotide molecule including all or a portion of a viral genome and an exogenous polynucleotide sequence. In some instances, gene transfer can be mediated by a DNA viral vector, such as an adenovirus (Ad) or adeno-associated virus (AAV). Other vectors useful in methods of gene therapy are known in the art. For example, a construct of the present disclosure can include an alphavirus, herpesvirus, retrovirus, lentivirus, or vaccinia virus. The exogenous sequences generally encode recombinant molecules to be expressed in the cells, e.g., for use in cell therapy. Processing steps of the methods can also or alternatively include all or a portion of cell washing, dilution, selection, isolation, separation, cultivation, stimulation, packaging, and/or formulation. The methods generally allow for the processing, e.g., selection or separation and/or transduction, of cells on a large scale (such as in compositions of volumes greater than or at about 50 mL).

In some embodiments, hematopoietic stem cells are genetically modified using gene editing applications which utilize site-specific nucleases for knock-out of targeted genomic sequences or knock-in of exogenous sequences, and for transferring exogenous sequences to the cells by viral transduction through the use of recombinant viral vectors. In some such embodiments, hematopoietic stem cells are collected by apheresis, enriched from the apheresis product, then cryopreserved prior to performing any gene editing method (e.g., gene knock-out, gene knock-in, gene correction). Cryopreservation may be introduced after mobilization and collection (e.g. by apheresis) of stem cells and selection for hematopoietic stem cells. Following cryopreservation, an assessment can be made on whether the threshold number of hematopoietic stem cells has been collected from the donor to proceed with the gene editing steps that follow. If a threshold number of cells has not been reached from a single round of mobilization, collection, selection and cryopreservation, subsequent rounds may be performed until the threshold number of cells has been reached. Threshold numbers of hematopoietic stem cells to be collected may vary depending on a number of factors, including but not limited to, the gene editing procedure performed (e.g., gene knock-out, gene knock-in, gene correction), the targeted gene to be edited, the mechanism by which the targeted gene is modified (e.g., homology dependent repair (HDR)), the efficiency of the editing procedure (e.g. HDR efficiency) and the therapeutic threshold for treatment of a specific disease. In some embodiments, the threshold number of hematopoietic stem cells to be collected from a donor prior to gene editing is about 1×10⁴ to 1×10⁵, 1×10⁵ to 1×10⁶, 1×10⁶ to 1×10⁷ cells/kg or more. In some embodiments, at least about 1×10⁵ to 1×10⁷ cells/kg are collected prior to gene editing. In some embodiments, at least about 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or about 1×10⁸ hematopoietic stem cells/kg are collected prior to proceeding with gene editing of the collected cells. Once the threshold number of hematopoietic stem cells are mobilized, collected, selected for, and cryopreserved, the cells can then proceed to thaw, culture and gene editing.

In some embodiments, the gene editing utilizes a nuclease introduced to the cell that is capable of causing a double-strand break near or within a genomic target site, which may be useful for increasing the frequency of homologous recombination and HDR at or near the cleavage site. In preferred embodiments, the recognition sequence for the nuclease is present in the host cell genome only at the target site, thereby minimizing any off-target genomic binding and cleavage by the nuclease. Gene-editing nucleases useful for the methods provided herein include but are not limited to a TAL-effector DNA binding domain-nuclease fusion protein (TALEN), a site-specific recombinase (for example, serine recombinase or a tyrosine recombinase, integrase (FLP, Cre, lambda integrase) or resolvase; a transposase, a zinc-finger nuclease (ZFN), and a clustered regularly interspaced short palindromic repeats (CRISPR) and CRISPR-associated (Cas) protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

In some embodiments, genetically modified CD34+ stem cells are generated by introducing a CRISPR-associated Cas nuclease (e.g. Cas9), a guide RNA polynucleotide, and a donor polynucleotide sequence into primary CD34+ stem cells. Through introduction of these components into the cell, a double stranded break can be introduced at a specific site as directed by the guide polynucleotide sequence and the CRISPR-associated Cas9 nuclease. A donor polynucleotide containing a sequence of interest can be further introduced into the cell and through homology directed recombination, the sequence of interest can be inserted into the cell. The transfer of the donor polynucleotide sequence can be carried out by transduction. The methods for viral transfer, e.g., transduction, generally involve at least initiation of transduction by incubating in a centrifugal chamber an input composition comprising the cells to be transduced and viral vector particles containing the vector, under conditions whereby cells are transduced or transduction is initiated in at least some of the cells in the input composition, wherein the method produces an output composition comprising the transduced cells.

Methods for introducing polypeptides, nucleic acids, and viral vectors (e.g., viral particles) into a primary cell, target cell, or host cell are known in the art. Any known method can be used to introduce a polypeptide or a nucleic acid (e.g., a nucleotide sequence encoding the DNA nuclease or a modified sgRNA) into a primary cell, e.g., a human primary cell. Non-limiting examples of suitable methods include electroporation (e.g., nucleofection), viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

In some embodiments the Cas nuclease can be in the form of a protein. In some embodiments, the Cas nuclease can be in the form of a plasmid, thereby allowing a cell that carries this expression construct to then express the Cas nuclease. In other embodiments, the Cas nuclease is pre-complexed with a guide RNA and introduced into the cell as a ribonucleoprotein (RNP). In some embodiments, the Cas nuclease and the guide polynucleotide sequence is introduced into the CD34+ cell through electroporation.

Introduction of the donor polynucleotide can occur through viral transduction using a delivery vector, such as adeno associated virus (AAV). AAV of any serotype or pseudotype can be used. Certain AAV vectors are derived from single stranded (ss) DNA parvoviruses that are nonpathogenic for mammals. Briefly, rep and cap viral genes that can account for 96% of the archetypical wild-type AAV genome can be removed in the generation of certain AAV vectors, leaving flanking inverted terminal repeats (ITRs) that can be used to initiate viral DNA replication, packaging and integration. Wild type AAV integrates into the human host cell genome with preferential site specificity at chromosome 19q13.3. Alternatively, AAV can be maintained episomally. At least twelve human serotypes of AAV (AAV serotype 1 (AAV-1) to AAV-12) and more than 100 serotypes from nonhuman primates have been discovered to date. Any of these serotypes, as well as any combinations thereof, may be used within the scope of the present disclosure. A serotype of the viral vector can be selected from the group consisting of AAV1, AAV2, AAV3, AAV4, AAV5, AAV6, AAV7, AAV8, and AAV9. In some embodiments, the serotype is AAV6.

In some embodiments, the viral transduction occurs within 30 minutes of the electroporation. In some embodiments, the viral transduction occurs simultaneously with the electroporation. In some embodiments, the viral transduction occurs within 1, 2, 3, 4, 5, 6, 7, 8, 9, 10, 15, 20, 25, or 30 minutes of the electroporation.

In other embodiments, hematopoietic stem cells are genetically modified using gene editing applications which utilize base editors. Base editing is a CRISPR-Cas9-based genome editing technology that allows the introduction of point mutations in the DNA without generating DSBs. Two major classes of base editors have been developed: cytidine base editors or CBEs allowing C>T conversions and adenine base editors or ABEs allowing A>G conversions (see e.g. Rees et al. (2018) Nat Rev Genet 19:770-788).

In other embodiments, hematopoietic stem cells are genetically modified using gene editing applications which utilize prime editors. Prime editors (PE) consist of nCas9 fused to a reverse transcriptase used in combination with a prime editing RNA (pegRNA, a guide RNA that includes a template region for reverse transcription). Prime editing allows introduction of insertions, deletions (indels) and 12 base-to-base conversions. Prime editing relies on the ability of a reverse transcriptase (RT), fused to a Cas nickase variant, to convert RNA sequence brought by a prime editing guide RNA (pegRNA) into DNA at the nick site generated by the Cas protein. The DNA flap generated from this process is then included or not in the targeted DNA sequence. See, e.g. Anzalone et al. (2019) Nature 576:149-157. Non-limiting examples of prime editing systems include PE1, PEI-M1, PE1-M2, PE1-M3, PE1-M6, PE1-M15, PE1-M3inv, PE2, PE3, PE3b.

### Dosing and Administration of Genetically Modified HSCs

In certain embodiments, the methods comprise administering to an individual in need of treatment a composition comprising an effective amount of genetically modified hematopoietic stem cells. Therapeutically effective doses of the hematopoietic stem cells can be in the range of about one million to about 200 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges. In some embodiments, the method comprises administering between 2×10⁶ and 2×10⁸ viable hematopoietic stem cells per kg of body weight.

In certain embodiments, pharmaceutical compositions comprising genetically modified hematopoietic stem cells in accordance with the present disclosure may be administered at dosage levels sufficient to deliver from, e.g., about 1×10⁴ to 1×10⁵, 1×10⁵ to 1×10⁶, 1×10⁶ to 1×10⁷, or more cells to the subject, or any amount sufficient to obtain the desired therapeutic or prophylactic, effect. The desired dosage of the modified host cell pharmaceutical compositions of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years. In some embodiments, only a single dose is needed to effect treatment or prevention of a disease or disorder described herein. In other embodiments, a subject in need thereof may receive more than one dose, for example, 2, 3, or more than 3 doses of a pharmaceutical hematopoietic stem cells compositions described herein to effect treatment or prevention of the disease or disorder. The hematopoietic stem cells may be used in combination with one or more other therapeutic, prophylactic, research or diagnostic agents, or medical procedures, either sequentially or concurrently. In general, each agent will be administered at a dose and/or on a time schedule determined for that agent.

The infusion population and compositions thereof can be administered to an individual in need thereof using standard administration techniques, formulations, and/or devices. Provided are formulations and administration with devices, such as syringes and vials, for storage and administration of the compositions. Formulations or pharmaceutical composition comprising exogenous hematopoietic stem cells include those for intravenous, intraperitoneal, subcutaneous, intramuscular, or pulmonary administration. Compositions of the exogenous hematopoietic stem cells can be provided as sterile liquid preparations, e.g., isotonic aqueous solutions, suspensions, emulsions, dispersions, or viscous compositions, which may in some aspects be buffered to a selected pH. Viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the hematopoietic stem cells in a solvent, such as in admixture with a suitable carrier, diluent, or excipient such as sterile water, physiological saline, glucose, dextrose, or the like.

Genetically modified hematopoietic stem cells included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously. The pharmaceutical compositions may be administered to a subject using any amount and any route of administration effective for preventing, treating, or managing a disease described herein. The exact amount required will vary from subject to subject, depending on the species, age, and general condition of the subject, the severity of the disease, the particular composition, its mode of administration, its mode of activity, and the like.

In some embodiments, the pharmaceutical composition comprises a modified host cell that is genetically engineered to comprise an integrated donor sequence at a targeted gene locus of the host cell. In some embodiments, the modified host cell is genetically engineered to comprise an integrated functional donor sequence, for example, a SNP donor that corrects one or mutations in a target gene (e.g. HBB) or inserts into or replaces some or all of the mutated allele with a wild-type allele. In particular embodiments, a functional donor sequence is integrated into the translational start site of the endogenous locus of the target gene. In particular embodiments, the functional donor sequence that is integrated into the host cell genome is expressed under control of the native promoter sequence of the target gene.

In some embodiments, the pharmaceutical composition comprises a plurality of the modified host cells, and further comprises unmodified host cells and/or host cells that have undergone nuclease cleavage resulting in INDELS at the target gene locus but not integration of the donor sequence. In some embodiments, the pharmaceutical composition is comprised of at least 5% of the modified host cells comprising an integrated donor sequence. In some embodiments, the pharmaceutical composition is comprised of about 9% to 50% of the modified host cells comprising an integrated donor sequence. In some embodiments, the pharmaceutical composition is comprised of at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50% or more of the modified host cells comprising an integrated donor sequence. The pharmaceutical compositions described herein may be formulated using one or more excipients to, e.g.: (1) increase stability; (2) alter the biodistribution (e.g., target the cells to specific tissues or cell types, e.g. hematopoietic stem cells); and/or (3) enhance engraftment in the recipient.

### Indications

The genetically modified HSCs described herein may be used as part of a treatment regimen for any disease or condition for which HSC transplantation (HSCT) is useful. HSCT may be used to treat a number of conditions, including congenital and acquired conditions. In some embodiments, acquired conditions treatable with HSCT include but are not limited to: (1) malignancies, including hematological malignancies such as leukemias (e.g. acute lymphoblastic leukemia (ALL), acute myeloid leukemia (AML), chronic lymphocytic leukemia (CLL), chronic myelogenous leukemia (CML)), lymphomas (e.g. Hodgkin's disease, Non-Hodgkin's lymphoma), myelomas (e.g. multiple myeloma (Kahler's disease)); solid tumor cancers (e.g. neuroblastoma, desmoplastic small round cell tumor, Ewing's sarcoma, choriocarcinoma); (2) hematologic disease, including phagocyte disorders (e.g. chronic granulomatous disease), bone marrow failure disorders (e.g. myelodysplastic syndrome, Fanconi's anemia, dyskeratosis congenita), anemias (e.g. paroxysmal nocturnal hemoglobinuria, aplastic anemia, acquired pure red cell aplasia), myeloproliferative disorders (e.g. polycythemia vera, essential thrombocytosis, myelofibrosis); (3) metabolic disorders including amyloidosis (e.g. amyloid light chain (AL) amyloidosis); (4) environmentally-induced diseases such as radiation poisoning; (5) viral diseases (e.g. HTLV, HIV); and (5) autoimmune diseases such as multiple sclerosis.

In some embodiments, congenital conditions treatable with HSCT include but are not limited to: (1) lysosomal storage disorders, including lipidoses (disorders of lipid storage, such as neuronal ceroid lipofuscinoses (e.g. infantile neuronal ceroid lipofuscinosis (INCL, Santavuori disease) and Jansky-Bielschowsky disease (late infantile neuronal ceroid lipofuscinosis)); sphingolipidoses (e.g. Niemann Pick disease and Gaucher disease), leukodystrophies (e.g. adrenoleukodystrophy, metachromatic leukodystrophy, Krabbe disease (globoid cell leukodystrophy); mucopolysaccharidoses (e.g. Hurler syndrome (MPS I H, α-L-iduronidase deficiency), Scheie syndrome (MPS I S), Hurler-Scheie syndrome (MPS I H-S), Hunter syndrome (MPS II, iduronidase sulfate deficiency), Sanfilippo syndrome (MPS III), Morquio syndrome (MPS IV), Maroteaux-Lamy syndrome (MPS VI), Sly syndrome (MPS VII)); glycoproteinoses (e.g. Mucolipidosis II (I-cell disease), fucosidosis, aspartylglucosaminuria, alpha-mannosidosis); and Wolman disease (acid lipase deficiency); (2) immunodeficiencies, including T-cell deficiencies (e.g. ataxia-telangiectasia and DiGeorge syndrome), combined T- and B-cell deficiencies (e.g. severe combined immunodeficiency (SCID), all types), well-defined syndromes (e.g. Wiskott-Aldrich syndrome), phagocyte disorders (e.g. Kostmann syndrome, Shwachman-Diamond syndrome), immune dysregulation diseases (e.g. Griscelli syndrome, type II), innate immune deficiencies (e.g. NF-Kappa-B Essential Modulator (NEMO) deficiency (Inhibitor of Kappa Light Polypeptide Gene Enhancer in B Cells Gamma Kinase deficiency)); (3) hematologic diseases, including hemoglobinopathies (e.g. sickle cell disease, thalassemia (e.g. β thalassemia)), anemias (e.g. aplastic anemia such as Diamond-Blackfan anemia and Fanconi anemia), cytopenias (e.g. Amegakaryocytic thrombocytopenia) and hemophagocytic syndromes (e.g. hemophagocytic lymphohistiocytosis (HLH)).

In some embodiments, the disease or condition is selected from the group consisting of a hemoglobinopathy, a viral infection, X-linked severe combined immune deficiency, Fanconi anemia, hemophilia, neoplasia, cancer, amyotrophic lateral sclerosis, alpha antitrypsin deficiency, Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders, bone or cartilage diseases and disorders, neurological and neuronal diseases and disorders, cardiovascular diseases and disorders, pulmonary diseases and disorders, and lysosomal storage disorders. In some embodiments, the hemoglobinopathy is selected from the group consisting of sickle cell disease, α-thalassemia, β-thalassemia, and δ-thalassemia.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLE

### Example 1: Single-Cell RNA Sequencing of Sickle Cell Reticulocytes to Identify Beta-Globin Genotypes and Associated Gene Expression Differences

A proof-of-concept single cell experiment was conducted with a mixture containing only wild type (AA) and sickle disease (SS) reticulocytes to demonstrate that HBB genotypes of individual cells can be clearly called using the custom bioinformatics pipeline described herein.

Methods: Reticulocytes were isolated from peripheral blood from healthy donors (AA), donors with sickle cell trait (AS), and donors with sickle cell disease (SS) using a combination of density-based enrichment and fluorescence-activated cell sorting (FACS) based on a Live/CD235a+/CD45−/TO+phenotype, as well as CD71 surface expression (FIG. 3). Portions of each isolated population were mixed at given ratios before based on cell counts, re-quantified and diluted to target loading amounts before application to the 10× genomics workflow for scRNAseq.

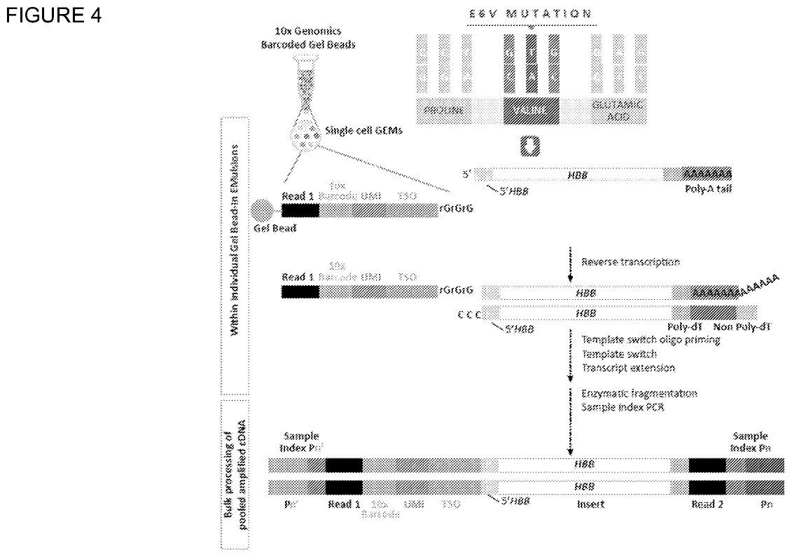

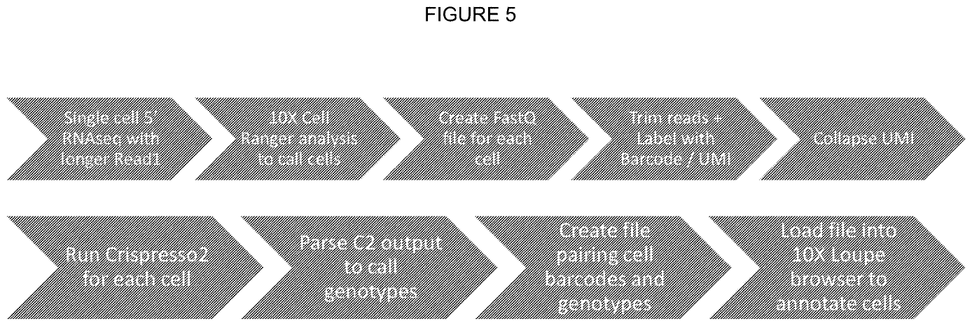

The 10× Genomics 5′ single-cell RNA sequencing kit was utilized to ensure adequate sequencing coverage across the 5′ end of HBB transcripts. The 10× scRNAseq workflow tags individual mRNA molecules with cell barcodes, as well as unique molecular identifiers (UMI) that enable transcript quantification. Sequencing is performed with a long Read 1 rather than the protocol outlined in the standard 10× Genomics scRNAseq protocol in order to sequence far enough into the 5′ end of HBB transcripts (FIG. 4). A custom bioinformatics analysis pipeline (FIG. 5) was developed to identify HBB variants within the scRNAseq data to identify individual reticulocytes expressing either normal HBB, sickle HBB, or both.

Results obtained with these scRNAseq methods were compared to those obtained with RNA bulk sequencing of individual sorted reticulocyte samples as well as blood and reticulocyte mixes. RNA was extracted from individual AA, AS, and SS reticulocyte samples, as well as even mixes of blood volumes and sorted reticulocytes. HBB transcripts were sequenced using a targeted cDNA sequencing assay. Briefly, RNA was converted to cDNA using a standard reverse transcription reaction. The region of HBB containing the edit site was PCR amplified, indexed, and sequenced.

### Results

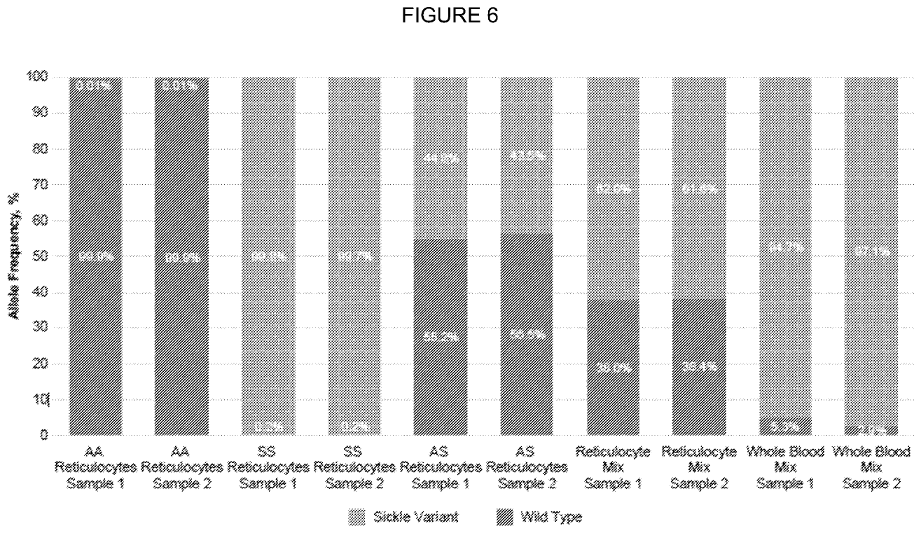

Bulk cDNA sequencing reproducibly estimates HBB allele content, but RNA content differs between AA and SS donors. HBB allele frequencies were reproducibly called using cDNA sequencing of reticulocyte pools and whole blood samples (FIG. 6). Bulk cDNA sequencing of reticulocyte and blood mixes could overestimate the number of SS cells. cDNA sequencing of pure AA and SS reticulocytes results in very clean and expected allele frequency calls. The AS sequencing results are slightly skewed towards the A allele. For the reticulocyte mix, even numbers of AA, AS, and SS reticulocytes were combined. The flow data showed that the SS reticulocytes had brighter thiazole orange staining indicative of a higher RNA content. The higher HBB transcript levels in the less mature SS reticulocytes result in an elevated S allele frequency for the reticulocyte mix. For the whole blood mix, the effect of SS reticulocytes is even more pronounced. The reticulocyte frequency in SS blood was 10 times higher than in AA or AS blood, and as such a mix of even blood volumes results in a very high S allele frequency.

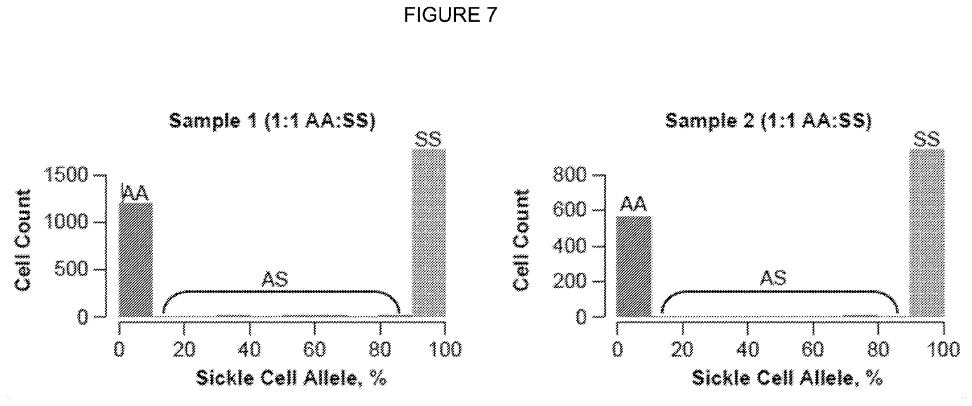

Single cell RNA sequencing estimates zygosity of AA/AS/SS alleles. To determine if the HBB genotypes of individual cells could be accurately called from scRNAseq data, a proof-of-concept, single-cell experiment was conducted with a mixture containing only wild-type (AA) and sickle disease (SS) reticulocytes to demonstrate that HBB genotypes of individual cells can be clearly called using a custom bioinformatics pipeline. Histograms of the sickle allele frequencies show that most cells either have 0% or 100% S allele frequency. Very few reticulocytes carry A and S alleles, indicative of mixed cells after single-cell sequencing (FIG. 7).

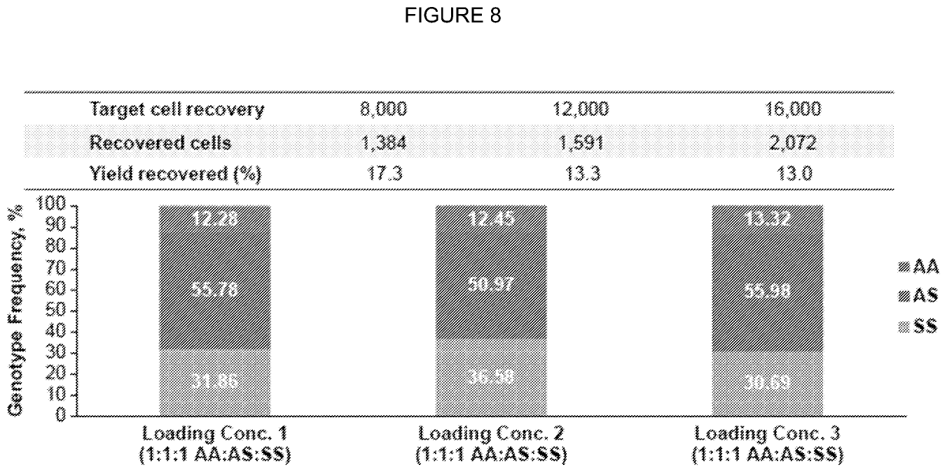

This approach was then applied to a mixture of AA, AS and SS reticulocytes. The 10× Genomics approach was performed using different loading concentrations to examine the impact on cell yield and genotyping. The percentage of cells recovered after single-cell sequencing was lower than anticipated based on other experiments with reticulocytes. Reticulocytes express fewer genes than most of the cells typically assessed by single-cell sequencing, but based on 10× Genomics QC metrics, such as percentage of reads assigned to cells, this appears to have little impact on cell identification. Overall, even the lowest input resulted in well over 1000 single cells identified and genotyped. Despite low recovery, results from these experiments show all 3 HBB genotypes can be reproducibly called using the scRNAseq 5′ protocol (FIG. 8) and are sufficient to assess editing zygosity and the impact editing might be having on a cell's gene profile.

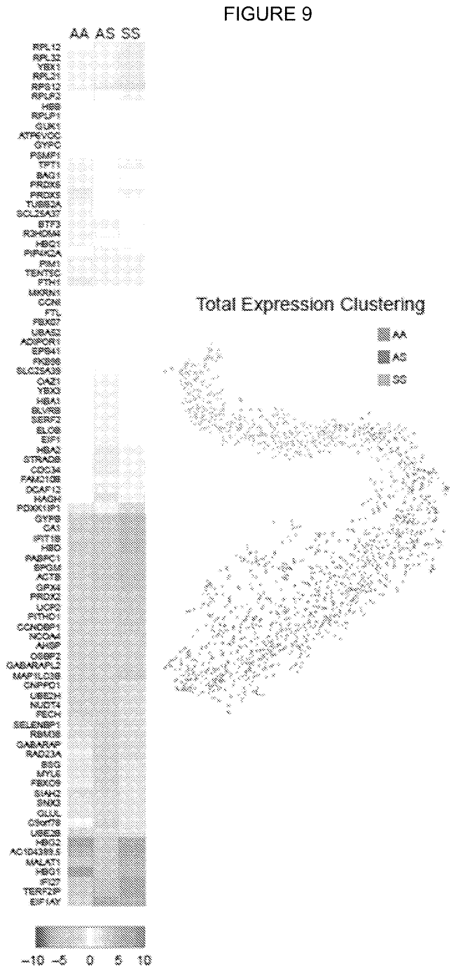

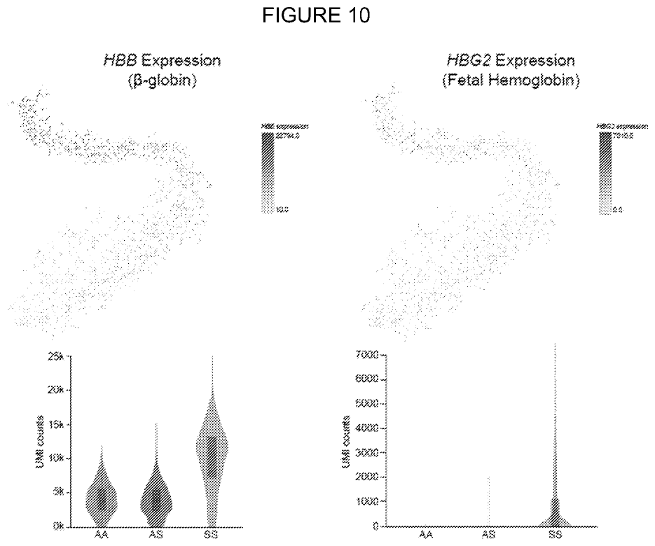

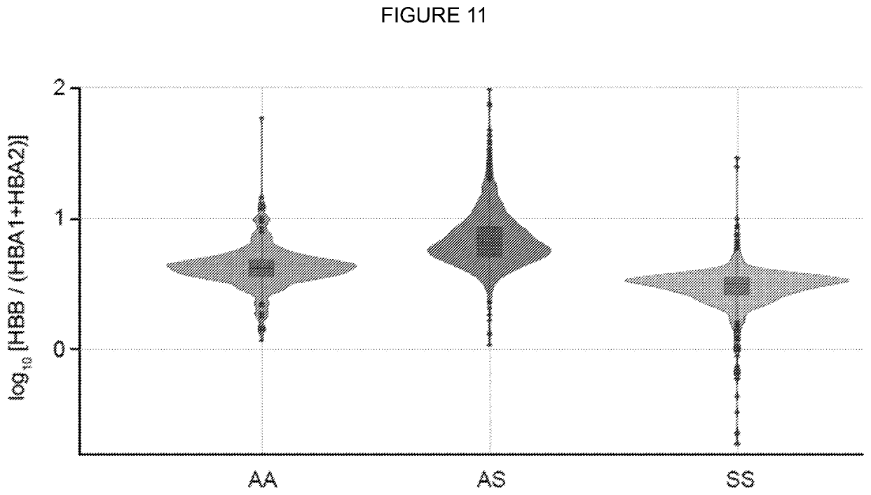

scRNAseg is able to differentiate SS from AA & AS reticulocytes. Overlaying the genotype calls on single-cell clustering results (t-SNE plot) shows that the SS reticulocytes form a separate population from the AA and AS reticulocytes. Differential gene expression analysis on the three genotypes also highlight the similarities of AA and AS reticulocytes and their marked difference from the SS reticulocyte population (FIG. 9). In comparison to AA and AS reticulocytes, SS reticulocytes had higher HBB, HBG2 (fetal hemoglobin), and overall higher transcript (UMI) counts (FIG. 10). Interestingly, both total and HBB transcript counts align with thiazole orange staining results, suggesting total UMI counts could be used to classify reticulocyte age/maturity. Although SS reticulocytes had higher total expression levels, their ratio of HBB to HBA1/HBA2 expression is lower than in AA and AS reticulocytes (FIG. 11).

Conclusion: These data demonstrate the utility of scRNAseq for evaluating differential HBB editing outcomes in erythroid progenitors from patients treated with gene-edited autologous hematopoietic stem cell-based therapies. The workflow and bioinformatic pipeline described herein enables genotype calling and classification of editing outcomes from single-cell RNA sequencing data. The results of this Example shows that this approach can call the HBB genotypes of individual reticulocytes, and that the method is amenable to determining HBB editing outcomes in erythroid progenitors. Additionally, this analysis enables correlation of genotype with gene expression profiles, as demonstrated by the distinct expression profiles of HbAA and HbAS versus HbSS cells. In principle, the approach described herein can be applied to any region of an expressed edited gene of interest via single-cell targeted RNA sequencing.

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

What is claimed:

1. A method of assessing gene-editing outcomes in a population of hematopoietic stem cells comprising:
a. isolating a population of reticulocytes from a subject having received a population of gene edited hematopoietic stem cells; and
b. genotyping the population of reticulocytes by quantifying RNA transcripts of single cells of the population of reticulocytes.

2. The method of claim 1, wherein the quantifying of RNA transcripts comprises single cell RNA sequencing.

3. The method of claim 1 or 2, wherein the gene-edited hematopoietic stem cells are autologous hematopoietic stem cells.

4. The method of any one of claims 1 to 3, wherein the gene-edited hematopoietic stem cells comprise CD34+ hematopoietic stem and progenitor cells (HSPCs).

5. The method of claim 4, wherein the CD34+ HSPCs comprise CD34+/CD38−/CD90+ HSPCs.

6. The method of claim 4, wherein the CD34+ HSPCs comprise CD34+/CD38−/CD90+/CD45RA− HSPCs.

7. The method of any one of claims 1 to 6, wherein genetic editing of the population of hematopoietic stem cells comprises contacting the population of hematopoietic stem cells with a CRISPR-associated Cas nuclease and a guide polynucleotide sequence that hybridizes to a target sequence in the hematopoietic stem cell genome.

8. The method of claim 7, further comprising contacting the population of hematopoietic stem cells with an AAV vector comprising a donor polynucleotide sequence.

9. The method of any one of claims 1 to 8, wherein the gene editing aims to correct a gene mutation, replace a mutant allele with a wild-type allele, or inserts a nucleic acid sequence encoding a therapeutic protein.

10. The method of any one of claims 1 to 9, wherein the subject suffers from a disease.

11. The method of claim 10, wherein the disease is a hemoglobinopathy.

12. The method of claim 11, wherein the hemoglobinopathy is selected from the group consisting of sickle cell disease, α-thalassemia, β-thalassemia, and δ-thalassemia.

13. The method of any one of claims 1 to 12, wherein the subject is human.
